(19) United States
(12) Patent Application Publication
(10) Pub. No.: US 2019/0262415 A1
(43) Pub. Date: Aug. 29, 2019

(54) Supplement for inducing and sustaining nutritional ketosis

(71) Applicant: Ketologie LLC, Dallas, TX (US)

(72) Inventors: Tracey Lee King, Richmond (NZ); James Tangihaere John Ehau, Richmond (NZ)

(21) Appl. No.: 16/286,442

(22) Filed: Feb. 26, 2019

Related U.S. Application Data
(60) Provisional application No. 62/635,480, filed on Feb. 26, 2018.

(51) Int. Cl. A61K 36/889; A61K 35/742; A61K 31/232; A61K 31/19; A61K 31/21; A61K 33/06; A61K 33/00; A61K 31/194; A61K 31/522; A23L 33/115; A23L 33/16; A23L 33/00; A23L 33/135

(52) U.S. Cl. CPC A61K 36/889; A61K 35/742; A61K 31/232; A61K 31/19; A61K 31/21; A61K 33/06; A61K 2035/115; A61K 31/194; A61K 31/522; A23L 33/115; A23L 33/16; A23L 33/40; A23L 33/135; A61K 33/00

(57) ABSTRACT

A dietary supplement for inducing and sustaining nutritional ketosis is disclosed. The invention addresses the many benefits of nutritional ketosis confers upon the human body via a composition containing exogenous ketones, ketone precursors, mineral cations, and probiotics. Such a combination improves metabolism, provides alternative fuels for the body under a diseased metabolic state, and strengthens digestion by supplementing the gut microbiome.

### CROSS-REFERENCE TO RELATED APPLICATIONS

The present application claims benefit to U.S. provisional application 62/635,480 filed on Feb. 26, 2018.

### BACKGROUND OF THE INVENTION

In the past 100 years or so, the health crises facing America have shifted from infectious disease and trauma to chronic (long-term) illnesses and disabilities. Metabolic syndrome, obesity, Type 2 diabetes, Alzheimers and other dementia, and chronic psychological disorders such as depression and anxiety are taking their toll on the health and well-being of the population.

Obesity and overweight conditions in particular have emerged in recent years as serious public health problems, affecting a combined estimated ⅔ of the population. Childhood obesity is also rapidly growing and has been identified as a top priority health concern for the nation.

Obesity can be defined as an abnormal or excessive fat accumulation in adipose tissue, to the extent that health is impaired. Obesity is considered to present significant health risks to the individual, being strongly correlated with greater risk of heart disease, high cholesterol and blood pressure, type 2 diabetes, stroke and breast, colon and prostate cancers.

It is possible that the over-consumption of starchy and sugary carbohydrates in the Standard American Diet (SAD) has contributed to these so-called “diseases of affluence.” The carbohydrate-insulin-obesity hypothesis postulates that carbohydrate consumption directly results in a surge of blood sugar, which the body treats as a medical emergency. High blood sugar is extremely dangerous and so insulin is immediately released to restore blood sugar to ‘normal’ levels. However, repeated exposure to insulin can cause insulin receptors to become burned out, a condition known as insulin resistance, the precursor to Type 2 diabetes.

Dietary regimes, such as Very Low Carbohydrate High Fat (VLCHF) or ketogenic ways of eating may prove to be a useful means to prevent insulin resistance and/or hyperinsulinemia from occurring or even reverse these conditions. When intake of starchy carbohydrates is restricted, stored glycogen is depleted and an alternative fuel source becomes a metabolic necessity. Under these metabolic conditions, ketone bodies (fatty acids) are produced for energy and the individual is said to be in a state of nutritional ketosis.

There are generally three types of ketones: beta-hydroxybutyrate (BHB), acetoacetate, and acetone, and other forms of ingestible ketones such as esters. They may also have the names 3-hydroxybutyrate or have the Greek alphabet beta (β) as a prefix, and are often referred to by the acronym BHB, βHB, BOHB, and various derivatives. The level of ketones present in the body, indicative of a physiologic state of nutritional ketosis, may be measured via blood, breath, or urine analysis.

Ketosis may be entered into either via restriction of dietary carbohydrate intake, by eating relatively high proportions of other macronutrients (fat and protein), by fasting, or by intense exercise (using up stored glycogen via energy expenditure). People who achieve a state of nutritional ketosis often report improved appetite control, reduced cravings for sugary foods, increased mental acuity or lifting of ‘brain fog’, reduced joint pain, better quality sleep, reduced sleep apnea, swifter decision-making, and improved sports performance (endurance). The broad metabolic and physiological advantages of elevating ketone levels and sustaining a state of ketosis has led many people to embrace considerable lifestyle changes.

However, not all people find it easy or convenient to follow the strict eating and exercise habits necessary for the body to stay in ketosis by usual means. In recent years, exogenous ketones, wherein ketones of an out-of-body (exogenous) origin are ingested as opposed to the endogenous or ketones that the body produces, have emerged as a convenient alternative for people wishing to experience some of the positive benefits of being in ketosis even when they have glycogen stores still available in their body.

Thus, there is a need for products or food/dietary supplement compositions that provide the benefits of nutritional ketosis and promote gut health, without the use of sugar or other detrimental sweeteners.

### BRIEF SUMMARY OF THE INVENTION

The present invention relates to a combination of exogenous ketones, ketone precursors (coconut oil or other forms of medium chain triglycerides such as MCT oil, and their derivative powders) and probiotics in a powdered dietary supplement that also is very low in carbohydrate, using only natural flavors and sweeteners that do not spike blood sugar or insulin.

In one embodiment, an arbitrary quantity of the present invention comprises 10-25%, by weight, coconut oil powder, compounds that yield digestible medium chain triglycerides, or other ketone precursors; 40-65% of ketone salts, such as sodium BHB, magnesium BHB, or calcium BHB; and 0.5-2.0% probiotic.

In another embodiment, in addition to the embodiment above, 20-30% by weight of the invention may be sodium BHB; 5-15% may be magnesium BHB; and 13-25% may be calcium BHB.

In another embodiment, an arbitrary quantity of the present invention comprises 10-25%, by weight, coconut oil powder, compounds that yield digestible medium chain triglycerides, or other ketone precursors; 40-65% of ketone ester; and 0.5-2.0% probiotic.

In another embodiment, in addition to the embodiment above, the invention may optionally contain potassium salts, such as potassium citrate; citric acid; non-sugar sweeteners such as Stevia, Erythritol or other sugar alcohols, or monk fruit extract; flavouring; natural colors.

In another embodiment, in addition to the embodiment above, the invention may optionally contain sufficient quantities of fiber or other substances that facilitate or help deliver probiotic bacteria to the colon, or themselves may digest in the colon to feed and enrich gut flora.

In another embodiment, in addition to the embodiment above, the invention may optionally contain 0.1-3.0% caffeine.

In another embodiment, one serving of the present invention comprises a mixed dry powder, 10-20 g by mass, which may be dissolve-able in drinking water in a preparation for consumption by the user.

In another embodiment, the invention is presented as a composition that can be combined with water for easy and convenient consumption, such that it is a powdered beverage mix.

In another embodiment, a composition comprising BHB, ketone precursors, and natural caffeine to act as a pre-workout supplement is disclosed.

In another embodiment, a composition comprising BHB, ketone precursors, and blends of nootropic substances, aimed at athletes to provide optimal building blocks for managing flow state with jitters is disclosed.

In another embodiment, a composition comprising BHB, ketone precursors, and inositol/L-theanine for neuro-rest and recovery is disclosed.

### DETAILED DESCRIPTION OF THE INVENTION

This invention relates to the novel use of exogenous ketone bodies in combination with resistant probiotic organisms to assist the body's transition into nutritional ketosis and to simultaneously support immune and digestive health. In one embodiment of the invention, the use of a combination of Bacillus coagulans GBI-30, coconut oil, and mineral salts of beta-hydroxybutyrate (βHB) is presented to provide an easy and gut-friendly method for inducing and sustaining nutritional ketosis.

One of the well-observed physiological consequences of nutritional ketosis (particularly while training the body to burn ketones rather than glucose for fuel) is that the body requires a substantially higher intake of mineral salts. If the individual who is taking action to put themselves in a state of nutritional ketosis does not at the same time increase their sodium, calcium, potassium and magnesium intake, they are prone to experiencing a range of unpleasant physical symptoms such as tiredness, achiness, muscle pain, headaches, irritability and more. Colloquially, this condition has become known as “keto flu” and is easily prevented or rectified simply by ensuring that these essential minerals are added into the individual's diet at sufficient (higher) levels. The invention seeks to allay the issue of potential mineral deficiency during keto-adaptation as the exogenous ketones are provided bonded to calcium, sodium and magnesium. Additional potassium is also included.

The invention discloses the use of probiotics in conjunction with exogenous ketones. Probiotics are beneficial bacteria that live in the human digestive tract and promote digestive and immune health. Probiotics are intended to bolster the body's naturally occurring intestinal flora and to help natural flora to maintain and/or re-establish themselves. Probiotics are sometimes recommended by doctors and nutritionists to aid in digestion, especially if the natural flora has become diseased through antibiotic treatments, illness, or by other reasons (such as a bad diet). However, many types of probiotics cannot survive the harsh acidic stomach environment.

There are ten times as many microbial cells in the human intestine as there are cells in the entire body, and those microbes play a role in a countless number of biological and chemical functions. Balance among the hundreds of species of bacteria in the gut is crucial to helping control pathogenic microbes that would cause a disease or hold potential pathogens under control. Dysbiosis occurs when the balance is disturbed and the concentrations of some commensal bacteria increase while others decrease.

Extensive scientific evidence now exists that points to the importance of gut microflora for good immune system function and overall health and well-being. A healthy and diverse diet is important to promote a well-functioning gut microbiota. Exemplary products manufactured by the present Applicant are made from “real” (whole) foods and therefore are naturally abundant in fiber; in addition to these important prebiotic building blocks, there are included 2 billion CFU's per serving in the present invention to support optimal digestive and immune system functioning.

There are many foods on the market that contain probiotics. However the majority are found in products that also contain high levels of sugar and other carbohydrates, such as flavored yoghurts and natural/organic kombucha drinks. Still many others do not contain strains of probiotics that can survive digestion by stomach acid.

The purpose of the current invention is to provide an alternative method for priming the gut with beneficial flora without consuming unnatural and unhelpful amounts of sugars or starches.

Bacillus coagulans is a lactic acid-producing bacterial species within the genus Bacillus. Bacillus coagulans is a Gram-positive rod (0.9 by 3.0 to 5.0 μm in size), catalase positive, spore forming, motile, and a facultative anaerobe. Bacillus coagulans is found in two forms, the vegetative form which is a regular bacterial cell similar in structure to other probiotic bacteria and the spore form, a naturally dormant form which can withstand many conditions such as heat, pressure, and extreme acid or alkaline conditions, any of which may kill vegetative bacterial cells.

One such resilient probiotic is Ganeden Biotech's GanedenBC³⁰ (Bacillus coagulans GBI-30, 6086). The composition of GanedenBC³⁰ differentiates itself from other probiotics by its ability to survive manufacturing processes, shelf life, stomach acids and intestinal bile. This allows GanedenBC³⁰ to be incorporated into a variety of foods and dietary supplements.

For a probiotic to be effective, it must survive the transit through the stomach and reach the intestines alive, where it confers a benefit. Using a validated gut model, it was demonstrated that GanedenBC³⁰ survives to populate and germinate in the intestines (Honda 2011a). When consumed daily, GanedenBC³⁰ has been shown in multiple studies to have a supportive effect on the digestive system. GBI-30 has been shown to decrease abdominal pain and bloating (Hun 2009), reduce the number of daily bowel movements (Dolin 2009) as well as decrease postprandial gas and bloating (Kalman 2009).

Research on GanedenBC³⁰ has shown that it supports immune system health in a variety of ways. One study found that it positively affected the actions of immune cells while also producing a cascade of cytokines, which are chemical messengers that coordinate the immune response, when after subjects had taken a daily dose of GanedenBC³⁰ for 30 consecutive days.

In additional studies GanedenBC³⁰ has been shown to have an immunomodulation effect on immune tissues (Jensen et al., 2010. Benson et al., 2012. Nyangale et al., 2014a). Besides a direct effect on the immune components of the intestine it has been recently demonstrated that this probiotic encourages the growth and maintenance of normal commensal bacteria known to be anti-inflammatory and whose presence is often drastically decreased in Crohn's disease and other forms of IBD (Nyangale et al., 2014; Sokol et al., 2008).

Many people use commercially prepared probiotic foods such as yoghurts or kombuchas in order to try to restore or maintain a diverse gut microflora. However, the majority of these contain high levels of sugar or other sweet/starchy carbohydrates such as fruit, fruit juice, honey, maple syrup, agave nectar, date syrup, rice malt syrup, cassava or tapioca syrup in order to overcome the naturally tart taste of fermented foods. In the context of the afore-mentioned metabolic health conditions, it would be desirable to be able to ingest probiotics but without incidentally spiking blood sugar and insulin.

It is important to note that all data relating to probiotic strains needs to be related directly to that strain and not simply the genus and or species. GanedenBC³⁰ has undergone extensive published safety testing and has even been proven safe when consumed at levels that far exceed standard food product inclusion levels (Endres 2009, Endres 2011). GanedenBC³⁰ has a GRAS (Generally Regarded as Safe) level of 93.6 billion CFU per day, and has received United States FDA GRAS status.

The following manufacturer claims are supported by third party research and are approved by the manufacturer of the probiotic ingredient:

“Supports digestive health” (at 1 billion CFU's per serve)

“Supports immune health” (at 500 million CFU's per serve)

“Supports and enhances protein utilization” (at 1 billion CFU's daily)

“Survives 10x more effectively than yogurt cultures”

While the use of the probiotic above is an embodiment of the invention, it is noted that the invention does not limit itself to just this form of probiotic. Other forms of probiotics, including prebiotic substances such as fiber that could be used to promote favorable gut flora growth, and other microbes that have similar resilient properties, may also be used.

Among sweeteners contemplated by the present invention, Erythritol is often referred to as a ‘benign’ sweetener by natural health practitioners and experts in the management of Type 2 diabetes and obesity, as it has negligible impact on blood sugar and insulin. Erythritol is a compound found in a variety of plants, the commercially available ingredients most commonly are extracted from corn and/or grapes.

Stevia is similarly a plant-based sweetener. Its high intensity lends it to being used in conjunction with other less-sweet base compounds (such as Erythritol); another non-sugar sweetener commonly used is monk fruit extract.

The present invention discloses a combination of exogenous ketones and probiotics because of the assertion that the brain and gut are intrinsically connected, and such an assertion is substantially supported by the scientific literature. The gut has an extensive network of neurons (the enteric nervous system) that communicates with the brain via what has become known as the gut-brain-axis. One mechanism by which this communication occurs is via hormonal signalling, whereby hormones released by the gut cross the blood-brain barrier, controlling one's desire for food. The gut hormone ghrelin for example, signals that one is hungry, and glucagon-like peptide (GLP-1) influences one's perception of satiety (tells us when one is full). These and other gut hormones act on the reward-signalling neural circuits in the brain; which may go some way towards explaining cravings and why food seems to “taste better” when one is hungry.

Also well-known is the fact that the gut is home to an entire ecosystem of bacteria (microbes) that play important metabolic and regulatory roles relating to digestion (uptake of nutrition), immune system function (controlling the impact of pathogens), and modulation of hormone and neurotransmitter production. This gut microbiome contains hundreds of trillions of bacteria that communicate with the enteric nervous system and the brain. The precise mechanism by which this occurs is not yet well articulated by the scientific community, where it is currently referred to as “black box connectivity”; meaning a complex and not clearly defined interplay between hormones, immune signalling molecules, metabolic pathways, and anatomical structures such as the vagus nerve.

Recent published studies with “germ-free” mice suggest that specific gut microbiome profiles can influence and alter brain function; and that the integrity of the blood-brain barrier itself may be impacted by the strength and the diversity of the gut microbiome. Since compositional changes in the gut microbiota have been observed to be correlated with inflammatory and metabolic disease, in turn a wide array of human health issues that are indivisible from the healthy functioning of the gut may be affected.

Probiotic supplementation works to increase the presence and the diversity of the gut microbiome; and may therefore assist with these functions of immunity and brain health. If emotional states such as anxiety and depressed mood can be influenced by the gut-brain axis as the literature suggests, it is feasible that efforts to repopulate the gut with appropriate probiotics could be beneficial for both immunity and healthy affect. Mood-related disorders could be in part the result of mis-firing or insufficient neurotransmitters, for example, which could be positively affected by an improvement in the veracity of the gut microbiome.

Neurotransmitters partly depend upon proper hormonal signalling, and so it seems likely that individuals with either an insufficient gut microbiome (due to overuse of antibiotics for example) or an unbalanced microbiome (gut dysbiosis) are at risk of a myriad of health consequences. It is this line of thinking that has inspired the considerable research currently being undertaken to investigate the possible connections between gut health and obesity, mental health, cognitive function, metabolic syndrome, insulin resistance, Type 2 diabetes, Alzheimer's and more.

Ketones such as beta-hydroxybutyrate similarly show the ability to cross the blood-brain barrier and it is interesting therefore to consider the potentially synergistic effects that may occur from the simultaneous supplementation of both probiotics and ketone precursors. It is proposed by the inventor that the metabolic and therapeutic potential of exogenous ketones may be optimized via the better functioning of a well-populated and diverse microbiome.

Ketones are produced in the liver under certain physiological conditions such as prolonged fasting, calorie restriction, low carb/high fat consumption, and extended intense exercise (or any combination of those). The induction of a hyperketotic state confers a number of metabolic and therapeutic advantages (both acute and chronic) to the individual, however the strict nature of ketogenic dieting is such that many people find it difficult to adhere to long-term. Exogenous ketone supplementation (ingestion of synthetic beta-hydroxybutrate and ketogenic precursors such as MCT oil or coconut oil) may provide a useful adjunct to those seeking to induce a state of hyperketonemia.

The premise of this invention is that a well-functioning gut-brain-axis may assist or enhance ketone bodies in the provision of their documented metabolic and physiologic effects (including but not limited to mitochondrial biogenesis, enhanced ATP production, increased adenosine levels, improved hormonal regulation and optimal neurotransmitter production, release and uptake e.g. serotonin, GABA, glutamate); and that by providing probiotics within the invention in conjunction with ketones and/or ketone precursors, maximal metabolic, neuro-pharmacological (psychobiotic) and neurophysiologic benefits may be conferred.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A composition for inducing and sustaining nutritional ketosis, comprising:
a ketone precursor;
exogenous ketone;
mineral cation; and
probiotic.

2. The composition of claim 1, wherein the ketone precursor is one or more of coconut oil powder, medium-chain triglycerides, or combination thereof.

3. The composition of claim 1, wherein the exogenous ketone is one or more of sodium beta-hydroxybutyrate, calcium beta-hydroxybutyrate, magnesium beta-hydroxybutyrate, or combination thereof.

4. The composition of claim 1, wherein the exogenous ketone is a ketone ester.

5. The composition of claim 1, wherein the exogenous ketone and mineral cation combines in ionic salt form when in dry powder form.

6. The composition of claim 1, wherein the mineral cation is one or more of sodium, calcium, magnesium, potassium, or combination thereof.

7. The composition of claim 1, wherein the probiotic is Bacillus coagulans.

8. The composition of claim 1, further comprising citric acid.

9. The composition of claim 1, further comprising citrate.

10. The composition of claim 1, further comprising non-sugar sweetener.

11. The composition of claim 10, wherein the non-sugar sweetener is one or more of Stevia, Erythritol, monk fruit, or combination thereof.

12. The composition of claim 1, further comprising a flavoring ingredient.

13. The composition of claim 1, further comprising a color ingredient.

14. The composition of claim 1, further comprising caffeine.
